(19) United States
(12) Patent Application Publication
(10) Pub. No.: US 2025/0171787 A1
(43) Pub. Date: May 29, 2025

(54) APTAMERS, PHARMACEUTICAL COMPOSITIONS THEREOF, AND METHODS FOR PREVENTING OR TREATING BCMA-ASSOCIATED DISEASES OR CONDITIONS

(71) Applicant: Aptapeutics Limited, Hong Kong (CN)

(72) Inventors: Yuen Yee Elysian CHOW, Hong Kong (CN); Pak Yan LAM, Hong Kong (CN)

(21) Appl. No.: 18/918,101

(22) Filed: Oct. 17, 2024

Related U.S. Application Data
(60) Provisional application No. 63/604,153, filed on Nov. 29, 2023.

(51) Int. Cl. C12N 15/115; A61K 9/00; A61K 31/4745; A61P 17/00

(52) U.S. Cl. CPC C12N 15/115; A61K 9/0019; A61K 31/4745; A61P 17/00; C12N 2310/16; C12N 2310/321; C12N 2310/3515

(57) ABSTRACT

In certain embodiments, provided herein are compositions, recombinant-nucleotides, kits and methods targeting or specific to BCMA that are useful for the prevention or treatment of BCMA-associated diseases such as chronic inflammatory skin diseases or conditions. In some embodiments, provided are aptamers comprising a nucleotide sequence at least about 80%, 85%, 90%, 95% or 100% sequence identity to any one of SEQ ID NOS.: 1-18, or a variant or a fragment thereof. Other example embodiments are described herein. In certain embodiments, the provided compositions, recombinant nucleotides, kits and methods are highly effective in treating chronic inflammatory skin diseases, easy to manufacture and store, and safer for use.

### CROSS-REFERENCE TO RELATED APPLICATION

This application claims benefit under 35 U.S.C. § 119 (e) of U.S. Provisional Application having Ser. No. 63/604,153 filed Nov. 29, 2023, the entire contents of which is/are hereby incorporated by reference herein.

### REFERENCE TO SEQUENCE LISTING

This application contains a sequence listing which has been submitted electronically in ST.26 (xml) format and is hereby incorporated by reference in its entirety. Said ST.26 copy, created on 24 Nov. 2023, is named “A069.0000.001.PROUS.xml” and is 23 kilobytes in size.

### FIELD OF INVENTION

This application relates to compositions, recombinant nucleotides, kits and methods for preventing or treating BCMA-associated diseases or conditions such as chronic inflammatory skin diseases or conditions. In particular, the application relates to compositions, recombinant nucleotides, kits and methods for preventing or treating BCMA-associated diseases or conditions such as chronic inflammatory skin diseases or conditions.

### BACKGROUND OF INVENTION

Chronic inflammatory skin disease continues to be a heavy burden for patients due to the impact on quality of life, and 20-25% of the world's population is estimated to be affected. Although there are many chronic inflammatory skin diseases, the two most common are eczema (atopic dermatitis) and psoriasis. Eczema primarily occurs in children and has a reduced incidence rate in older populations. There are cases of adult-onset eczema, the incidence rate is lower. In contrast, psoriasis is primarily seen in adults. Both diseases result from inflammation caused by immune dysfunction. Genotypes and environmental stimuli, such as infection, allergens, stress, drugs, and trauma, have been implicated as potential causes of these diseases. These stimuli cause the expression of chemokines and cytokines, leading to infiltration by immune cells. Immune cell infiltration, followed by exposure to the inflammatory environment, causes these new immune cells to activate and further contribute to the inflammatory event. In turn, this leads to a rampant inflammatory feedback loop and severely affects patients' quality of life.

Multiple therapies are available for attempting to treat or prevent eczema and psoriasis, and they act by targeting various aspects of the disease. The first line of therapy for both involves corticosteroids. From a broad perspective, corticosteroids affect gene transcription via various mechanisms and different clinical benefits. Both methotrexate and acitretin inhibit the thickening of skin associated with eczema and psoriasis. Ciclosporin blocks the production of cytokines to reduce T cell activation, a key driver of the inflammation cascade. Dexamethasone suppresses immune cell migration, thereby reducing the number of cells involved in inflammation. Another corticosteroid, desonide, blocks gene transcription to reduce the expression of inflammatory proteins.

Small molecule inhibitors are another class of drugs used in combination with existing first line therapies, or as a second line therapy. Small molecule inhibitors (SMI) target proteins associated with the inflammation cellular signaling cascade. By binding to a protein and inhibiting its activity, dysregulation of the downstream signaling pathway occurs followed by alterations in the gene expression profile. SMIs are easily administered in the oral form, but systemic side effects are a concern, as the drug circulates throughout the body.

Another therapeutic option is monoclonal antibodies. These treatment plans rely on the modulation of the immune system via the use of monoclonal antibodies. The monoclonal antibodies have a high binding specificity for cytokine receptors, to block receptor-ligand interactions, or inflammatory cytokines. In both cases, the inflammatory activity of cytokines is inhibited. Although the response rate to monoclonal antibodies treatment is remarkably high, long-term treatment runs the risk of infusion reactions or increased infection risk.

Many current drug developments for chronic skin inflammation are focused on the inhibition of specific targets in the inflammation signaling cascade. This can be performed by inhibiting specific enzymes involved in cellular signaling, or via targeting the cytokines. All the available therapeutic methods have limitations, mostly due to the occurrence of side effects over long term use. As such, there exists an urgent need of targeting using different methods to target aspects of the inflammation cascade.

Chronic inflammatory skin diseases are characterized by a feedback loop involving cytokines. These cytokines attract immune cells to the region, then induce the expression of inflammatory cytokines, attracting additional immune cells. Inflammation leads to the development of skin lesions. Due to the feedback loop, there are many therapeutic targets within this milieu.

Targeting the inflammation cascade can occur in different ways. One current method is via the use of monoclonal antibodies. Antibodies are proteins with a high binding specificity and affinity for a ligand. The ligands can either be a cytokine, to prevent the signaling between cells, or a receptor, to block a cell's response to cytokine stimulation. Binding to a receptor has two further mechanisms: inhibiting the ability of the receptor to interact with its ligand or induce the internalization of the receptor so it is not available to bind to a ligand.

One of the issues with available therapies is the development of side effects after long term use. Long term topical corticosteroid application may lead to epidermal thinning, increased capillary permeability, ulceration, and other visible side effects. For all treatment options, one of the concerns is bacterial or viral infection. The immune response to infection or injury is for inflammation to occur. A systemic drug, such as an oral small molecule inhibitor or monoclonal antibodies, targets the inflammation cascade and blunts the response to infection. In addition, infusion reactions may occur during monoclonal antibody administration. Due to these issues, there is a need for an alternative treatment strategy which could be used over a long duration and have minimal potential side effects.

### SUMMARY OF INVENTION

Disclosed herein are novel compositions, recombinant nucleotides, sense and anti-sense strand thereof and sequences thereof, kits and methods targeting or specific to BCMA that are useful for the prevention or treatment of BCMA-associated diseases such as chronic inflammatory skin diseases or conditions.

In some embodiments, provided herein are compositions and methods for the highly selective delivery of nucleic acid sequences for the purpose of alleviating skin inflammation. More specifically, in some embodiments, provided herein are single stranded DNA aptamers comprising nucleic acid sequences to target B-cell maturation cell antigen (BCMA, CD269, TNFRSF17) expressed on the cell surface. By targeting the cell surface BCMA, the aptamer can inhibit inflammation associated with chronic skin inflammation. In some embodiment, provided are aptamers comprising comprises a nucleotide sequence with a sequence identity of at least 80% to any one of SEQ ID NOS.: 1-18, or a fragment thereof.

There are many advantages of the invention. In some embodiments, provided aptamers are very stable as they can be stored for extended periods in a lyophilized state. As aptamers are chemically synthesized, it is simple to scale up any production process, whilst maintaining a lower manufacturing cost than antibodies. The size of provided aptamers provides a major benefit when it comes to therapeutic use, as the injection volume required is much less due to its high solubility. In terms of safety, provided aptamers shows a lower toxicity in vitro.

Described herein is an invention describing a single stranded DNA (ssDNA) molecule for treatment of chronic skin inflammation. In particular, the molecule consists of an aptamer for targeting the cells of interest in vivo. Also provided herein are methods for the treatment of chronic skin inflammation by targeting BCMA expressing cells.

In another embodiment, the invention suppresses or attenuates the gene expression of desired cells via an aptamer DNA molecule wherein the molecule has a high affinity to cells expressing a unique cell surface marker and expresses cellular products associated with inflammation. The desired cells can range from keratinocytes, dendritic cells, macrophages, T cells, etc. The cellular products may include gene or protein targets such as one of the following: TSLP, IL-1β, IL-6, IL-19, IL-36a, IL-36b, IL-22, GM-CSF, TNF-α, IL-4, IL-33, IL-13, IL-23A, IL-17A, IL-17C, IL-17F, LCN2, CD31, VEGF-A, Ang-1, Ang-2, S100A8, S100A9, MCL-1.

In another embodiment, a method of administering a therapeutic dose of ssDNA aptamer, wherein the aptamer has a high binding affinity and is specific for BCMA and can bind to the BCMA expressed on the cell surface. The BCMA expressing cells may include skin tissue associated cells of the following subtypes: keratinocytes, dendritic cells, plasma cells and macrophages.

In another embodiment, a therapeutic dose of the BCMA targeting aptamer inhibits the expression of various inflammatory cytokines expressed in the skin tissue during inflammation. Examples of these cytokines may be one of the following: TSLP, IL-1β, IL-6, IL-19, IL-36a, IL-36b, IL-22, GM-CSF, TNF-α, IL-4, IL-33, IL-13, IL-23A, IL-17A, IL-17C, and IL-17F.

In one aspect, the aptamer can target BCMA and is specific for BCMA expressing cells. The ssDNA aptamer has the sequence SEQ ID NO.: 1 to SEQ ID NO.: 18. Aptamer candidates were identified via bead-based SELEX and were screened to select the sequences with the best BCMA binding activity. Upon binding to BCMA, the aptamer may inhibit the BCMA ligand signaling pathway, or induce the internalization of the aptamer-BCMA complex.

In one aspect, the BCMA targeting aptamer may have various nucleotide substitutions and one or more modifications to the sugar phosphate backbone to confer nuclease resistance. The nucleoside substitutions may comprise at least one or combinations thereof, of uracil, inverted dT, purine, xanthine, diaminopurine, 8-oxo-N6-methyladenine, 7-deazaxanthine, 7-deazaguanine, N4,N4-ethanocytosin, N6,N6-ethano-2,6-diaminopurine, 5-methylcytosine, 5-(C3-C6)-alkynylcytosine, 5-fluorouracil, 5-bromouracil, pseudoisocytosine, 2-hydroxy-5-methyl-4-triazolopyridin, isocytosine, isoguanine, inosine, non-naturally occurring nucleobases, variants, mutants and analogs thereof. Preferably, the sugar phosphate backbone modifications include 2′-O-methylation, locked nucleic acids (LNA), peptide nucleic acids (PNA), phosphorothioate, boranophosphate, methyl phosphate, 2′fluoro, 2′-O-methoxyethyl, or 4′-thio.

The BCMA targeting aptamer may have one or more chemical structures attached in order to increase serum half life, cellular internalization, or inhibit nuclease activity. Preferably, the additional chemical structure comprises at least one of the following: cholesterol, cholesteryl, inverted dT, phosphatidyl choline, phosphatidyl ethanolamine, spermine, sialic acid, histidyl poly(lysine), palmityl-D-glucuronide, polyethylene glycol, polyrotaxanes, chlorogenic acid chitosan, listeriolysin O, or tyloxapol. Preferably, the additional chemical structure(s) is conjugated through one or more of the following spacers or linkers: C12 spacer, C9 spacer, C6 spacer, C3 spacer, spacer 18 (hexaethylene glycol), spacer 9, dSpacer, rSpacer, amino linkers, carboxy linkers, or thiol linkers.

### BRIEF DESCRIPTION OF FIGURES

FIG. 1 shows results of an enzyme linked oligonucleotide assay (ELONA), where aptamer candidates were tested for BCMA binding specificity.

FIG. 2 is a set of plots showing the binding affinity for three selected example aptamers: SEQ ID NOS.: 1-3, as generated by ELONA.

FIG. 3 is a pair of images of denaturing polyacrylamide gel showing the stability of the example aptamers (SEQ ID NO.: 3) after incubation in mouse serum or culture medium+10% FBS for the indicated amount of time.

FIG. 4 is a set of images of denaturing polyacrylamide comparing the stability of an unmodified and modified version of example aptamer based on SEQ ID NO.: 6 (example modified aptamer is represented by SEQ ID NO.: 16) after incubation in fetal bovine serum for the incubated duration of time.

FIG. 5 is a plot showing the binding affinity plot for a modified example aptamer SEQ ID NO.: 16, as determined by ELONA.

FIG. 6 is a graph depicting the gene expression levels of select macrophage markers when an example aptamer of SEQ ID NOS.: 2-3 were used to inhibit the immunostimulatory effect of LPS.

FIG. 7 is a graph depicting the Psoriasis Area Severity Index (PASI) in an IMQ induced mouse model of psoriasis after subcutaneous treatment with a modified example aptamer based on SEQ ID NO.: 3 (modified example aptamer is represented by SEQ ID NO.: 17) or SEQ ID NO.: 6 (modified example aptamer is represented by SEQ ID NO.: 18).

FIG. 8 is a graph depicting the change in skin thickness of mice skin in an IMQ induced mouse model of psoriasis after subcutaneous treatment with a modified example aptamer SEQ ID NO.: 17 or SEQ ID NO.: 18.

FIG. 9 is a series of representative photos of the dorsal skins of mice. IMQ induced mouse model of psoriasis were administrated subcutaneously with vehicle or a modified example aptamers of SEQ ID NO.: 17 or SEQ ID NO.: 18.

FIG. 10 is a graph depicting the Eczema Area Severity Index in a DNFB induced mouse model of eczema. Example aptamers of SEQ ID NO.: 17 and SEQ ID NO.: 18 were given as a subcutaneous treatment option.

FIG. 11 is a series of representative images in a DNFB induced mouse model of eczema. The DNFB induced mice were administrated subcutaneously with vehicle or a modified example aptamers SEQ ID NO.: 17 or SEQ ID NO.: 18.

FIGS. 12A-12B is a series of graphs showing the frequency and duration of itch response in a DNFB induced mouse model of eczema. Modified example aptamers SEQ ID NO.: 17 and SEQ ID NO.: 18 were given as a subcutaneous treatment option to two groups of mice.

FIG. 13 is a graph showing the mRNA expression of IL-1b, TNFα and IL-33 in a DNFB induced model of eczema when example aptamers SEQ ID NO.: 17 and SEQ ID NO.: 18 were given as a subcutaneous treatment option.

FIG. 14 is a graph showing relative number of mast cells counted in the tissue section from a DNFB induced model of eczema when example aptamers SEQ ID NO.: 17 and SEQ ID NO.: 18 were given as a treatment option.

### DETAILED DESCRIPTION

As used herein and in the claims, the terms “comprising” (or any related form such as “comprise” and “comprises”), “including” (or any related forms such as “include” or “includes”), “containing” (or any related forms such as “contain” or “contains”), means including the following elements but not excluding others. It shall be understood that for every embodiment in which the term “comprising” (or any related form such as “comprise” and “comprises”), “including” (or any related forms such as “include” or “includes”), or “containing” (or any related forms such as “contain” or “contains”) is used, this disclosure/application also includes alternate embodiments where the term “comprising”, “including,” or “containing,” is replaced with “consisting essentially of” or “consisting of”. These alternate embodiments that use “consisting of” or “consisting essentially of” are understood to be narrower embodiments of the “comprising”, “including,” or “containing,” embodiments.

For example, alternate embodiments of “a composition comprising A, B, and C” would be “a composition consisting of A, B, and C” and “a composition consisting essentially of A, B, and C.” Even if the latter two embodiments are not explicitly written out, this disclosure/application includes those embodiments. Furthermore, it shall be understood that the scopes of the three embodiments listed above are different.

For the sake of clarity, “comprising”, including, and “containing”, and any related forms are open-ended terms which allows for additional elements or features beyond the named essential elements, whereas “consisting of” is a closed end term that is limited to the elements recited in the claim and excludes any element, step, or ingredient not specified in the claim.

For the sake of clarity, “characterized by” or “characterized in” (together with their related forms as described above), does not limit or change the nature of whether the list of terms following it are open or closed.

As used herein, the singular forms “a”, “an” and “the” are intended to include the plural forms as well, unless the context clearly indicates otherwise. Where a range is referred in the specification, the range is understood to include each discrete point within the range. For example, 1-7 means 1, 2, 3, 4, 5, 6, and 7.

As used herein, the term “about” is understood as within a range of normal tolerance in the art and not more than +10% of a stated value. By way of example only, about 50 means from 45 to 55 including all values in between. As used herein, the phrase “about” a specific value also includes the specific value, for example, about 50 includes 50.

As used herein and in the claims, an “effective amount”, is an amount that is effective to achieve at least a measurable amount of a desired effect. For example, the amount may be effective to elicit cell death. For example, the amount may be effective to elicit an immune response, and/or it may be effective to elicit a protective response, against a pathogen bearing the polypeptide of interest. In some embodiments, the amount may be effective to elicit an immune response against cancer or tumor.

As used herein and in the claims, a “subject” refers to animals such as mammals, including, but not limited to, primates (e.g., humans), cows, sheep, goats, horses, dogs, cats, rabbits, rats, mice and the like. In some examples, the subject refers to mouse or human.

As used herein, the term “treat,” “treating” or “treatment” refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

As used herein and in the claims, the term “prevent”, “preventing”, “preventive”, “preventative” or “prevention” refers the methods of reducing the risk of the onset, relapse or spread of a disease or disorder or one or more of their symptoms.

As used herein, the term “pharmaceutical composition” or “composition” refers to a formulation containing one or more active pharmaceutical ingredient(s).

As used herein, the term “variant sequence” refers to a nucleic acid or polypeptide sequence that displays certain degree of identity to a reference or wild-type nucleic acid or polypeptide sequence, for example, at least 80%, at least 85%, at least 90%, at least 95%, at least 96%, at least 97%, at least 98% or at least 99%. For example, a variant sequence has one or more additions, deletions, insertions, and/or substitutions or other modifications when compared to a reference sequence.

As used herein and in the claims, the term “pharmaceutically acceptable carrier” or “carrier” refers to a molecule or substance such as a protein or a nucleotide used as a vehicle or medium to deliver a drug or active ingredient in a pharmaceutical formulation. In some examples, the pharmaceutically acceptable carrier is conjugated to one or more active ingredients such as polypeptides.

As used herein and in the claims, the term “aptamer” or “aptamers” refers oligonucleotide(s) such as single stranded deoxyribonucleic acid (ssDNA), ribonucleic acid (RNA), xeno nucleic acid (XNA), or peptide that binds a specific target molecule, or family of target molecules (such as proteins, peptides, and small molecules), which exhibit high affinities to the target molecule(s). In some examples, the aptamer is a ssDNA that binds or targets specifically to BCMA.

As used herein and in the claims, the terms “substitution”, “substitute” refers to the process of replacing one or more nucleotides (e.g., adenine, guanine, cytosine, or thymine/uracil) with a different nucleotide(s) within the aptamer sequence, such as to improve the aptamer's binding affinity, stability, or other desirable properties.

As used herein and in the claims, the terms “modification” or “modify” refers to the process of changing (such as adding, deleting, substituting) chemical groups or molecules to one or more nucleotides within the aptamer sequence. In some examples, these modifications can enhance the aptamer's properties, such as improved stability, target binding, or detection capabilities, without changing the core sequence.

As used herein and in the claims, the term “disease or condition” refers to a state of being or health status of a patient or subject capable of being treated with a compound, pharmaceutical composition, or method provided herein. In some examples, the disease or condition refers to BCMA-associated diseases or conditions such as chronic inflammatory skin diseases, disorders or conditions.

As used herein and in the claims, the terms “identical” or percent “identity,” in the context of two or more nucleic acids or polypeptide sequences, refer to two or more sequences or subsequences that are the same or have a specified percentage of amino acid residues or nucleotides that are the same (e.g., about 80%, 85%, 90%, 91%, 92%, 93%, 94%, 95%, 96%, 97%, 98%, 99%, or higher identity over a specified region, when compared and aligned for maximum correspondence over a comparison window or designated region).

By way of example, provided aptamers or compositions described herein is administered by any suitable route of administration. A route of administration may refer to any administration methods known in the art, including but not limited to aerosol, enteral, nasal, ophthalmic, oral, parenteral, rectal, transdermal (e.g., topical cream or ointment, patch), or vaginal. Transdermal administration may be accomplished using a topical cream or ointment or by means of a transdermal patch. Parenteral refers to a route of administration that is generally associated with injection, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. In some embodiments, provided aptamers or compositions described herein are administered by intravenous injection or intraperitoneal injection.

As used herein and in the claims, B cell maturation cell surface marker (BCMA, CD269, or TNFRSF17) is a member of the tumor necrosis factor superfamily. It is a non-glycosylated integral membrane receptor for the ligands B cell activating factor (BAFF) and a proliferation inducing ligand (APRIL).

Although the description referred to particular embodiments, the disclosure should not be construed as limited to the embodiments set forth herein.

The invention relates to compositions and methods of delivering a BCMA targeting aptamer to BCMA-expressing cells to inhibit specific aspects of inflammation. The present invention provides an agent comprising of a BCMA specific aptamer.

In one embodiment, the invention describes aptamers. An aptamer is a nucleic acid molecule with a high binding affinity and specificity for a target molecule of interest. They are identified through SELEX (Systemic Evolution of Ligands by Exponential enrichment). The SELEX process involves processing multiple rounds of binding, selection, and amplification of an oligonucleotide library to identify the oligonucleotide with a high specific binding to the desired target. Although high specificity oligonucleotides may have been identified, it does not guarantee a desired binding affinity, or the same profile in a cell rich environment. In the embodiments described herein, SELEX selection was utilized to identify aptamers with a high binding affinity and specificity for BCMA.

In some embodiments, the previously identified aptamers underwent truncation, backbone modifications, or sugar base modification. These modifications to the aptamers were verified for increased binding specificity. According to prior art, these modifications provide an additional benefit of increasing exo-/endonuclease resistance of the aptamer. These modifications increase the half-life of the aptamer, and act to prolong the duration of therapeutic effect.

According to the embodiments described herein, aptamers specific for BCMA may bind to cells expressing BCMA, which is a cell surface receptor. The primary ligand of BCMA is a proliferation-inducing ligand (APRIL), which then initiates a signaling cascade in MM cells. This signaling cascade leads to proliferation, differentiation, and survival via canonical and non-canonical NFkB2 activation (Kleber 2021). Although BCMA is expressed by mature B lymphocytes and plasma cells, BCMA expression has also been identified in the skin lesions of psoriasis and eczema patients (Alexaki 2012, Carpenter 2013, Tai 2014, Leyva-Castillo 2020).

In some embodiments, the aptamer and BCMA interaction may inhibit the induction of the BCMA signaling pathway. This may occur by blocking the interaction between APRIL and BCMA, or by inducing the internalization of BCMA. Other aptamers have shown that interaction with BCMA leads to rapid internalization of the cell surface protein (Catuogno 2019). Through the internalization effect, the reduced presence of BCMA on the cell surface reduces the stimulatory effects of its ligands.

In some embodiments, the binding of the aptamer to BCMA inhibited the activity of NFκB. NFκB plays a significant role in the inflammation response and acts as a transcription factor for multiple inflammatory cytokines. These cytokines elicit a response from other innate immune cells, which then act upon the adaptive immune cells. The creation of an inflammatory environment leads to a positive feedback loop wherein rampant inflammation leads to issues for the patient.

In some embodiments, the aptamer reduces the inflammation present in skin lesions.

### NUMBERED EMBODIMENTS

### Set 1

Embodiment 1. An aptamer, wherein the aptamer comprises a nucleotide sequence with a sequence identity of at least 80% to any one of SEQ ID NOS.: 1-18, or a fragment thereof.

Embodiment 2. The aptamer of embodiment 1, wherein the nucleotide sequence comprises or consist of SEQ ID NO.: 3, SEQ ID NO.: 6, SEQ ID NO.: 16, SEQ ID NO.: 17 or SEQ ID No.: 18.

Embodiment 3. The aptamer of any one of the preceding embodiments, wherein the nucleotide sequence comprises one or more of the following nucleoside substitutions: uracil, inverted dT, purine, xanthine, diaminopurine, 8-oxo-N6-methyladenine, 7-deazaxanthine, 7-deazaguanine, N4,N4-ethanocytosin, N6,N6-ethano-2,6-diaminopurine, 5-methylcytosine, 5-(C3-C6)-alkynylcytosine, 5-fluorouracil, 5-bromouracil, pseudoisocytosine, 2-hydroxy-5-methyl-4-triazolopyridin, isocytosine, isoguanine, inosine, non-naturally occurring nucleobases, variants, mutants, or analogs thereof.

Embodiment 4. The aptamer of any one of the preceding embodiments, wherein the nucleotide sequence comprises one or more of the following sugar phosphate backbone modifications: 2′-O-methylation, locked nucleic acids (LNA), peptide nucleic acids (PNA), phosphorothioate, boranophosphate, methyl phosphate, 2′fluoro, 2′-O-methoxyethyl, or 4′-thio.

Embodiment 5. The aptamer of any one of the preceding embodiments, wherein the nucleotide sequence is conjugated to one or more of the following chemical structures: cholesterol, cholesteryl, inverted dT, phosphatidyl choline, phosphatidyl ethanolamine, spermine, sialic acid, histidyl poly(lysine), palmityl-D-glucuronide, polyethylene glycol, polyrotaxanes, chlorogenic acid chitosan, listeriolysin O, or tyloxapol.

Embodiment 6. The aptamer of embodiment 6, wherein the nucleotide sequence is conjugated to the one or more chemical structures through one or more of the following spacers or linkers: C12 spacer, C9 spacer, C6 spacer, C3 spacer, spacer 18 (hexaethylene glycol), spacer 9, dSpacer, rSpacer, amino linkers, carboxy linkers, or thiol linkers.

Embodiment 7. A pharmaceutical composition, comprising: the aptamer of any one of the preceding embodiments; and optionally, a pharmaceutically acceptable carrier.

Embodiment 8. A method for preventing or treating a chronic inflammatory skin disease, disorder, or condition, comprising: administering an effective amount of the aptamer of any one of the embodiments 1-6, or the composition of embodiment 7, to a subject in need thereof.

Embodiment 9. The method of embodiment 8, wherein the chronic inflammatory skin diseases, disorder, or condition is selected from a group consisting of eczema and psoriasis.

### Set 2

Embodiment 1. An aptamer, wherein the aptamer comprises a nucleotide sequence with a sequence identity of at least about 80%, 85%, 90%, 95% or 100% sequence identity to any one of SEQ ID NOS.: 1-18, or a variant or a fragment thereof.

Embodiment 2. The aptamer of embodiment 1, wherein the nucleotide sequence comprises or consist of SEQ ID NO.: 3, SEQ ID NO.: 6, SEQ ID NO.: 16, SEQ ID NO.: 17 or SEQ ID No.: 18.

Embodiment 3. The aptamer of any one of the preceding embodiments, wherein the nucleotide sequence comprises one or more of the following nucleoside substitutions: uracil, inverted dT, purine, xanthine, diaminopurine, 8-oxo-N6-methyladenine, 7-deazaxanthine, 7-deazaguanine, N4,N4-ethanocytosin, N6,N6-ethano-2,6-diaminopurine, 5-methylcytosine, 5-(C3-C6)-alkynylcytosine, 5-fluorouracil, 5-bromouracil, pseudoisocytosine, 2-hydroxy-5-methyl-4-triazolopyridin, isocytosine, isoguanine, inosine, non-naturally occurring nucleobases, variants, mutants, or analogs thereof.

Embodiment 4. The aptamer of any one of the preceding embodiments, wherein the nucleotide sequence comprises one or more of the following sugar phosphate backbone modifications: 2′-O-methylation, locked nucleic acids (LNA), peptide nucleic acids (PNA), phosphorothioate, boranophosphate, methyl phosphate, 2′fluoro, 2′-O-methoxyethyl, or 4′-thio.

Embodiment 5. The aptamer of any one of the preceding embodiments, wherein the nucleotide sequence is conjugated to one or more of the following chemical structures: cholesterol, cholesteryl, inverted dT, phosphatidyl choline, phosphatidyl ethanolamine, spermine, sialic acid, histidyl poly(lysine), palmityl-D-glucuronide, polyethylene glycol, polyrotaxanes, chlorogenic acid chitosan, listeriolysin O, or tyloxapol.

Embodiment 6. The aptamer of embodiment 1, wherein the nucleotide sequence is conjugated to the one or more chemical structures through one or more of the following spacers or linkers: C12 spacer, C9 spacer, C6 spacer, C3 spacer, spacer 18 (hexaethylene glycol), spacer 9, dSpacer, rSpacer, amino linkers, carboxy linkers, or thiol linkers.

Embodiment 7. The aptamer of any one of the preceding embodiments, wherein the nucleotide sequence comprises the following modifications: a substitution of cytosines with 2′-O-methylation, and an addition of an inverted dT at the 3′ terminus.

Embodiment 8. The aptamer of any one of the preceding embodiments, wherein the nucleotide sequence comprises the following modifications: a substitution conjugation of a cholesteryl at the 5′ terminus through a spacer C9 and an inverted dT at the 3′ terminus.

Embodiment 9. A pharmaceutical composition, comprising: the aptamer of any one of the embodiments 1-8; and optionally, a pharmaceutically acceptable carrier.

Embodiment 10. A method for preventing or treating BCMA-associated diseases or conditions, comprising: administering an effective amount of an aptamer or a composition thereof, wherein the aptamer comprises a nucleotide sequence with a sequence identity of at least about 80%, 85%, 90%, 95% or 100% sequence identity to any one of SEQ ID NOS.: 1-18, or a variant or a fragment thereof, to a subject in need thereof.

Embodiment 11. The method of embodiment 10, wherein the nucleotide sequence comprises or consist of SEQ ID NO.: 3, SEQ ID NO.: 6, SEQ ID NO.: 16, SEQ ID NO.: 17 or SEQ ID NO.: 18.

Embodiment 12. The method of embodiment 10 or 11, wherein the composition comprises a pharmaceutically acceptable carrier.

Embodiment 13. The method of any one of the embodiments 10 to 12, wherein BCMA-associated diseases or conditions are chronic inflammatory skin diseases, disorders, or conditions.

Embodiment 14. The method of any one of the embodiments 10 to 13, wherein the chronic inflammatory skin diseases, disorders, or conditions is selected from a group consisting of eczema and psoriasis.

Embodiment 15. The method of any one of the embodiments 10 to 14, wherein the aptamer or the composition thereof is administered by subcutaneous injection.

Embodiment 16. The method of any one of the embodiments 10 to 15, further comprising the step of: topically administering an effective amount of imiquimod (IMQ) after the step of administering an effective amount of an aptamer or a composition thereof.

Embodiment 17. Use of an aptamer or a composition thereof in the manufacture of a medicament for preventing or treating BCMA-associated diseases or conditions in a subject, wherein the aptamer comprises a nucleotide sequence with a sequence identity of at least about 80%, 85%, 90%, 95% or 100% sequence identity to any one of SEQ ID NOS.: 1-18, or a variant or a fragment thereof.

Embodiment 18. An aptamer or a composition thereof for use in the prevention or treatment of BCMA-associated diseases or conditions in a subject, wherein the aptamer comprises a nucleotide sequence with a sequence identity of at least about 80%, 85%, 90%, 95% or 100% sequence identity to any one of SEQ ID NOS.: 1-18, or a variant or a fragment thereof.

### EXAMPLES

Provided herein are examples that describe in more detail certain embodiments of the present disclosure. The examples provided herein are merely for illustrative purposes and are not meant to limit the scope of the invention in any way. All references given below and elsewhere in the present application are hereby included by reference.

### Example 1 Identification of BCMA Specific Aptamers

The initial ssDNA library used consisted of nucleotides that are 71-nucleotide (nt) in length. The 35-nt random region in the middle is flanked by two 18-nt adapter regions. These adapter regions acted as primer binding sites for PCR amplification.

Selection of the ssDNA aptamers by bead bound SELEX involved multiple selection rounds. Each round involves a pre-selection step, selection step, and a regeneration step. A total of 20 rounds of SELEX were used. In all rounds, the positive selection step involved the immobilization of His-tagged recombinant human BCMA on Ni-NTA magnetic beads. The ssDNA pool was then incubated with the magnetic bead complex. The mixture was washed to remove unbound aptamers, and the bound aptamers were then eluted. The elution products were used in PCR with biotinylated reverse primers for amplification and quality checking via agarose gel electrophoresis.

For regeneration of the ssDNA pool, the PCR products were incubated with streptavidin coated magnetic beads, to bind the biotinylated reverse primers. Following incubation, the dsDNA PCR products were denatured by alkaline treatment. The resulting pool of ssDNA in the supernatant was then used for the next round of SELEX.

From round 4 onward, negative selection was performed prior to positive selection. Negative selection involved the incubation of the ssDNA pool with blank Ni-NTA beads or His-tagged non-target proteins for negative selection purposes. After incubation, the unbound ssDNA was then incubated with His-tagged recombinant BCMA-bound Ni-NTA beads.

The enriched ssDNA pools from Rounds 3, 6, 9, 12, 15, and 20, were modified by the VAHTS Universal DNA Library Prep Kit for Illumina® V3, according to the manufacturer's instructions, to ligate a 5′-universal adapter and a 3′ indexed DNA adapter to the ssDNA sequences. Prior to Next Gen Sequencing of the ssDNA pool, the concentration and size distribution of the library was measured with an Agilent 2100 Bioanalyzer with the High sensitivity DNA kit.

Enzyme-linked oligonucleotide assay (ELONA) was used to evaluate aptamer candidates. The aptamers were synthesized with a 5′ biotinylation modification. 500 ng of His-tagged recombinant BCMA was used to coat each well of a 96-well microtiter plate via overnight incubation at 4° C. 1 hour of BSA blocking at room temperature was used to cover non-binding sites and was followed by 4 washes with DNA binding buffer for 5 minutes. 1 μM of each aptamer was added into each well for 45-minute incubation with gentle shaking. The wells were then washed with wash buffer 4 times, 5 minutes each. 100 μl of streptavidin-HRP (1:10000 in PBST+0.1% BSA) was added into each well for 30-minute incubation. 4 washes with binding buffer were then performed. 50 μl of TMB was added to each well for a 20-minute incubation. 50 μl of 2M H₂SO₄ was used to stop the reaction, followed by measurement of 450 nm absorbance to determine specificity.

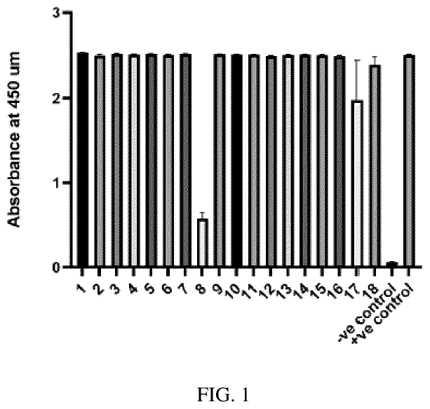

18 aptamer candidates were identified after various rounds of bead-bound SELEX. As observed in FIG. 1, evaluation of the binding specificity revealed that two of the aptamers did not have as strong of a binding specificity to the target protein. Of the candidates, only one did not have binding specificity to BCMA. Nucleotide sequences of samples 1, 7, and 10 were referred to as SEQ ID NOS.: 1, 2 and 3, respectively. Table 1 is a list of sequence information corresponding to Samples 1, 7 and 10 of Example 1.

TABLE 1
Sequence listing of Samples (example aptamers)
1, 7 and 10 (i.e. SEQ ID NOS .: 1, 2 and 3)
of Example 1

| | SEQ | |
|---|---|---|
| Sample | ID | |
| No. | NO. | Sequence |
| 1 | 1 | CGTACGGTCGACGCTAGCAAGTATAGACGTGATGC |
| | | AGGGAATGCAGACCCAGGGGATCCGAGCTCCACG |
| | | TG |
| 7 | 2 | CGTACGGTCGACGCTAGCCCATGGGAATGCAGACC |
| | | GCAGGCTGGCTGTAGGAGGGATCCGAGCTCCACG |
| | | TG |
| 10 | 3 | CGTACGGTCGACGCTAGCTGTTGCTGTGCAGGAAG |
| | | AATGCAGATTCCCAACATGGATCCGAGCTCCACGT |
| | | G |

### Example 2 Aptamer Affinity Evaluation

ELONA was used to evaluate the binding affinity of the aptamers. 500 ng of His-tagged recombinant BCMA was used to coat each well of a 96-well microtiter plate via overnight incubation at 4° C. 1 hour of BSA blocking at room temperature was used to cover non-binding sites and was followed by 4 washes with DNA binding buffer for 5 minutes. Varying concentrations of each aptamer was added into each well for 45-minute incubation with gentle shaking. The wells were then washed with wash buffer 4 times, 5 minutes each. 100 μl of streptavidin-HRP (1:10000 in PBST+0.1% BSA) was added into each well for 30-minute incubation. 4 washes with binding buffer were then performed. 50 μl of TMB was added to each well for a 20-minute incubation. 50 μl of 2M H₂SO₄ was used to stop the reaction, followed by measurement of 450 nm absorbance.

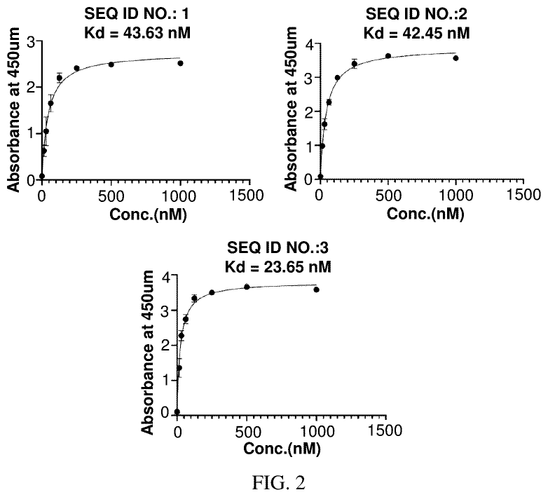

Binding affinity evaluation was performed for all aptamers, other than candidate 8. The absorbance was used to plot a binding curve, from which the binding affinity was determined for samples (example aptamers) 1, 7, 10 (SEQ ID NO.: 1-3) (FIG. 2).

### Example 3 Aptamer Serum Stability

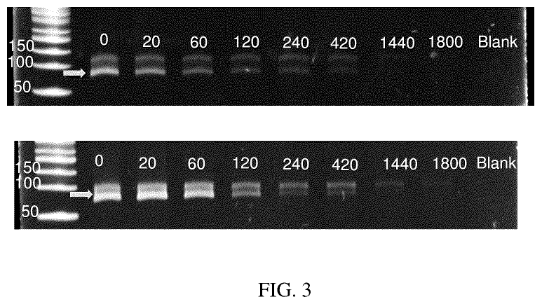

To test the serum stability of the aptamers, 20 nM samples 1, 7, 10 (SEQ ID NO.: 3) was incubated in mouse serum, or culture medium+10% FBS at 37° C. Samples were collected at the indicated time points and stored until evaluation (FIG. 3). Samples were diluted 100×, then were amplified with a Phusion High-Fidelity DNA Polymerase. After amplification was completed, the samples were loaded into a 15% denaturing polyacrylamide gel for electrophoresis. The gel was run, then stained with GelRed for imaging. Band densitometry analysis was performed after imaging.

As can be seen, the half-life of sample 10 SEQ ID NO.: 3 in mouse serum was around 1 hour, whilst the half-life in culture medium+10% fetal bovine serum was approximately 2 hours (FIG. 3).

### Example 4 Aptamer Truncation Evaluation

12 truncated example aptamers (SEQ ID NOS.: 4-15) were synthesized based on sample 10 SEQ ID NO.: 3. Table 2 shows the sequence listing of the 12 truncated aptamers (SEQ ID NOS.: 4-15) from sample 10 SEQ ID NO.: 3.

| | SEQ | |
|---|---|---|
| Sample | ID | |
| No. | NO. | SEQUENCE |
| 10-1 | 4 | CGTACGGTCGACGCTAGCTGTTGCTGTGCAGGAAG |
| 10-2 | 5 | CGTACGGTCGACGCTAGCTGTTGCTGTGCAGGAAGAATGC |
| | | A |
| 10-3 | 6 | CGTACGGTCGACGCTAGCTGTTGCTGTGCAGGAAGAATGC |
| | | AGATTC |
| 10-4 | 7 | CGTACGGTCGACGCTAGCTGTTGCTGTGCAGGAAGAATGC |
| | | AGATTCCCAAC |
| 10-5 | 8 | CGTACGGTCGACGCTAGCTGTTGCTGTGCAGGAAGAATGC |
| | | AGATTCCCAACATGGA |
| 10-6 | 9 | CGTACGGTCGACGCTAGCTGTTGCTGTGCAGGAAGAATGC |
| | | AGATTCCCAACATGGATCCGA |
| 10-7 | 10 | CGTACGGTCGACGCTAGCTGTTGCTGTGCAGGAAGAATGC |
| | | AGATTCCCAACATGGATCCGAGCTCC |
| 10-8 | 11 | TGTTGCTGTGCAGGAAGAATGCAGATTCCCAACAT |
| 10-9 | 12 | GGTCGACGCTAGCTGTTGCTGTGCAGGAAGAATGCAGATT |
| | | CCCAACATGGATCCGAGCTCCACGTG |
| 10-10 | 13 | ACGCTAGCTGTTGCTGTGCAGGAAGAATGCAGATTCCCAA |
| | | CATGGATCCGAGCTCCACGTG |
| 10-11 | 14 | AGCTGTTGCTGTGCAGGAAGAATGCAGATTCCCAACATGG |
| | | ATCCGAGCTCCACGTG |
| 10-12 | 15 | TTGCTGTGCAGGAAGAATGCAGATTCCCAACATGGATCCG |
| | | AGCTCCACGTG |

Their binding affinity was evaluated via ELONA. Binding curves were plotted, followed by calculation of the binding affinity. Table 3 shows the Kd values for all truncated aptamer sequences (SEQ ID NOS.: 4-15), with sample 10-3 SEQ ID NO.: 6 having the best Kd of all the truncated aptamers at 59 nM.

TABLE 3
Binding affinity of example aptamers (SEQ ID NOS.:
4-15) truncated from Sample 10 SEQ ID NO.: 3.

| SEQ ID NO. | Kd (nM) |
|---|---|
| 4 | 1292 |
| 5 | 240 |
| 6 | 59 |
| 7 | 130 |
| 8 | 579 |
| 9 | 364 |
| 10 | 65 |
| 11 | 184 |
| 12 | 451 |
| 13 | 1548 |
| 14 | 1592 |
| 15 | 542 |

### Example 5 Aptamer Modification

The serum stability was also examined for unmodified example aptamer (SEQ ID NO.: 6) and modified example aptamer based on SEQ ID NO.: 6 (modified example aptamer is represented by SEQ ID NO.: 16). The modification included the substitution of cytosines with 2′-O-methylation and the addition of an inverted dT at the 3′ terminus.

The sequence of the modified example aptamer (SEQ ID NO.: 16) is shown as below:

mCGTAmCGGTmCGAmCGmCTAGmCTGTTGmCTGTGmCAGGAAGAATG
mCAGATTmC-InvdT,

wherein m=2′-O-methylation; InvdT=3′ Inverted dT.

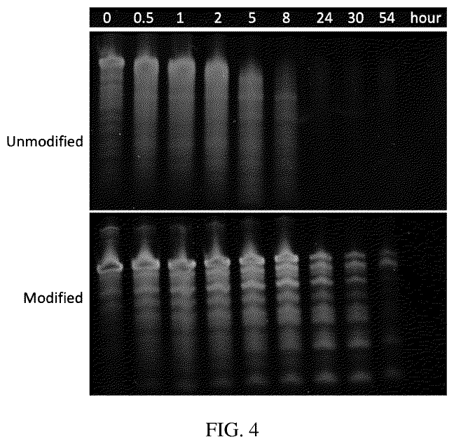

The aptamers were incubated in fetal bovine serum at 37° C. for the indicated time points (FIG. 4). At each time point, samples were collected and stored for later evaluation. The samples were run on a 20% denaturing polyacrylamide gel (FIG. 4). The gel was stained with SYBR gold for imaging.

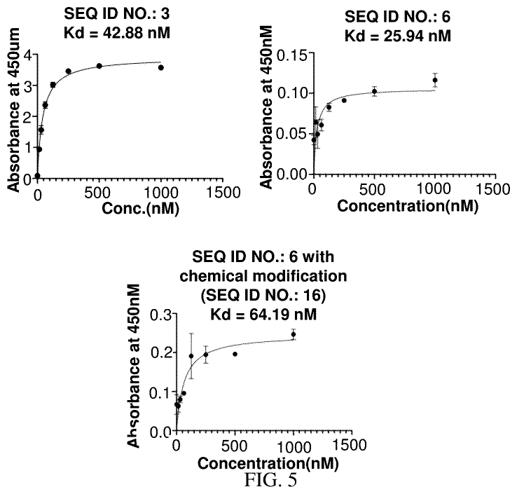

As can be seen in FIG. 4, the unmodified SEQ ID NO.: 6 was fully degraded after 8 hours, whilst some of the modified SEQ ID NO.: 6 was present at 54 hours. In addition to the increased half-life, binding affinity analysis of modified SEQ ID NO.: 6 showed that its Kd was around 64.19 nM (FIG. 5). This was performed via ELONA.

### Example 6 Inhibition of Inflammation Response

1.5×10⁵ RAW 264.7 murine macrophages were seeded into wells overnight. The medium was changed to 1% FBS in DMEM. Cells were treated with 300 nM of SEQ ID NO.: 2 or SEQ ID NO.: 3 for 15 minutes, then the cells were exposed to 1 μg/mL of LPS for 24 hours. After 24 hours, the RNA was extracted with TRIzol for qPCR. 24 hours of exposure to 1 μg/mL LPS stimulated the expression of iNOS, TNFα, CD163 and VEGFA mRNA in RAW264.7 cells.

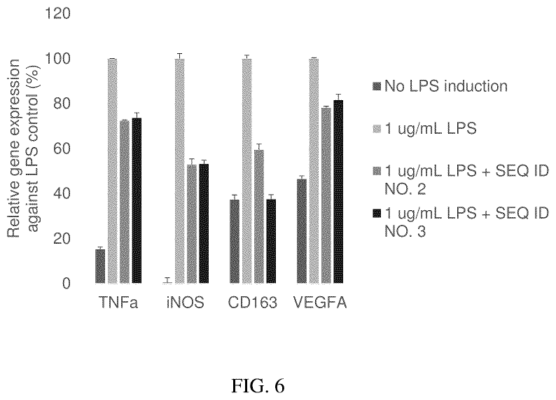

Macrophage exposure to LPS induces the M1 response which favors inflammation. Results showed that a 15-minute pre-treatment with 300 nM of SEQ ID NO.: 2 or 3 reduced the expression of iNOS, TNFα, CD163 and VEGFA. Here, the pre-treatment with BCMA-targeting aptamer sequences suppressed the induction of iNOS and TNF-α mRNA expression (FIG. 6). Both genes are associated with the M1 inflammatory response.

### Example 7 IMQ Mouse Model of Psoriasis

This animal study was approved by the Hong Kong Polytechnic University Animal Subjects Ethics Review (20-21/274-OTHERS-R-OTHERS). This animal study was performed at the Hong Kong Polytechnic University Shenzhen Research Institute animal.

On Day −1, the dorsal skin had its fur removed. On Day 0, the thickness of the shaved dorsal skin of the mice was measured via micrometer, and the skin was evaluated for Psoriasis Area Severity Index (PASI) scoring. Then the mice were given a subcutaneous injection of a 100 μL of buffer containing 600 pmol BCMA targeting aptamer, or buffer alone. Following administration of the aptamer, 62.5 mg of Aldara cream (5% IMQ) was topically applied to the shaved dorsal skin. On Days 1-3, the dorsal skin was evaluated for PASI scoring prior to the administration of the test-article, which was then followed by IMQ administration. On Day 4, the dorsal skin thickness was measured, and the PASI evaluation was performed prior to termination of the animals. Skin samples were collected and stored in RNAlater for later TRIzol RNA extraction and qPCR. The difference was calculated by subtracting the thickness measured at the beginning of experiment from that at the end of the experiment.

Mice were given modified example aptamer based on SEQ ID NO.: 3 (modified example aptamer is represented by SEQ ID NO.: 17) or SEQ ID NO.: 6 (modified example aptamer is represented by SEQ ID NO.: 18). The modifications included the substitution conjugation of a cholesteryl at the 5′ terminus through a spacer C9 and an inverted dT at the 3′ terminus.

The sequences of the modified example aptamers SEQ ID NOS.: 17-18 are shown as below:

(SEQ ID NO .: 17)
Cholesteryl-SpC9-
CGTACGGTCGACGCTAGCTGTTGCTGTGCAGGAAGAATGCAGATTCCCAA
CATGGATCCGAGCTCCACGTG-InvdT
(SEQ ID NO .: 18)
Cholesteryl-SpC9-
CGTACGGTCGACGCTAGCTGTTGCTGTGCAGGAAGAATGCAGATTC-
InvdT

wherein SpC9=Spacer C9 and InvdT=3′ Inverted dT.

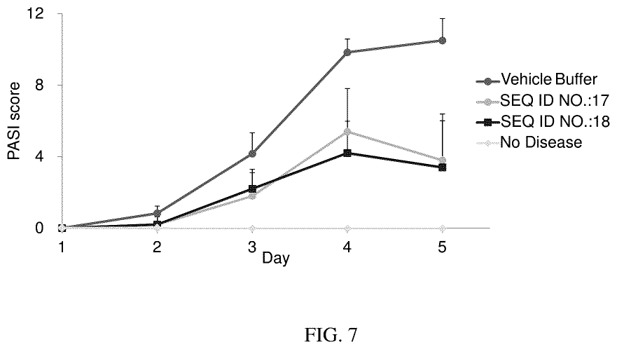

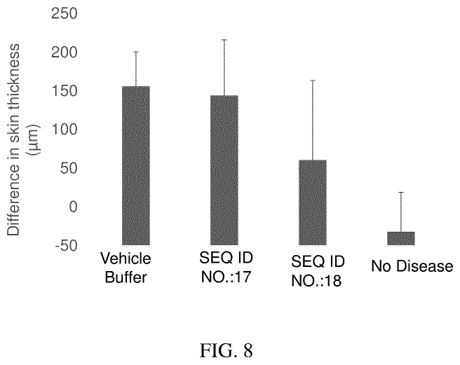

The aptamers were given as a treatment option in conjunction with IMQ, the aptamer sequences inhibited the development of the psoriasis phenotype (FIG. 7). Aligned with the differences in the psoriasis phenotype, there was significant differences observed in change of skin thickness of SEQ ID NO.: 18 treated mice (FIG. 8). Results showed that SEQ ID NO.: 18 reduced the associated increase in skin thickness.

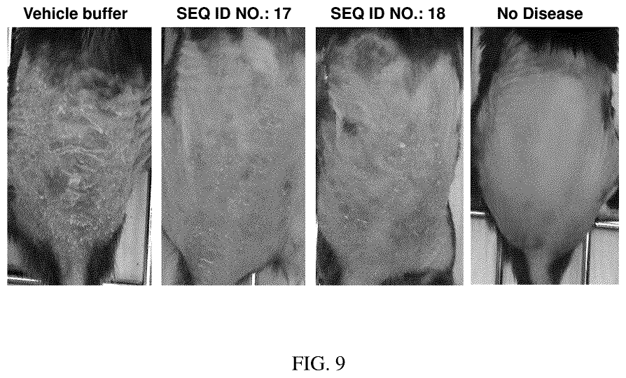

FIG. 9 contains a set of representative images of the skin.

The qPCR results showed a similar overall trend. IMQ treatment led to the induction of various markers (S100A8, S100A9, KRT6B, KRT16) consistent with the development of psoriasis (see Table 4). Treatment with SEQ ID NO.: 17 or 18 ameliorated the expression of these psoriasis associated markers. In addition to the amelioration of psoriasis markers, BCMA targeting aptamer treatment also reduced the expression of multiple inflammatory cytokines, such as TSLP, IL-1β, IL-6, IL-19, IL-17C, IL-17F (Table 4). These cytokines all have a role in initiating an innate or adaptive immune response.

TABLE 4
Relative expression (% against vehicle buffer group) of select
psoriasis markers in an IMQ induced model of psoriasis

| | | SEQ ID | SEQ ID | |
|---|---|---|---|---|
| | Vehicle buffer | NO.: 17 | NO.: 18 | No Disease |
| IL-6 | 100.0 | 65.7 | 36.1 | 5.9 |
| TSLP | 100.0 | 59.7 | 47.9 | 2.2 |
| LCN2 | 100.0 | 80.8 | 49.2 | 3.1 |
| AREG | 100.0 | 73.2 | 50.0 | 20.5 |
| S100A8 | 100.0 | 90.8 | 53.1 | 0.1 |
| S100A9 | 100.0 | 71.9 | 54.8 | 0.1 |
| IL-19 | 100.0 | 90.1 | 54.8 | 10.4 |
| IL-1b | 100.0 | 70.4 | 57.5 | 11.9 |
| KRT6b | 100.0 | 60.9 | 65.3 | 0.8 |
| KRT16 | 100.0 | 65.4 | 65.6 | 1.7 |
| IL-17c | 100.0 | 103.9 | 70.8 | 7.5 |
| IL-17a | 100.0 | 108.7 | 76.5 | 109.8 |
| IL-17f | 100.0 | 112.1 | 77.8 | 48.9 |
| iNOS | 100.0 | 87.8 | 77.9 | 87.6 |

Results showed that both SEQ ID NO.: 17 and SEQ ID NO.: 18 were capable of ameliorating the development of skin lesions.

### Example 8 DNFB Mouse Model of Eczema

This animal study was approved by the Hong Kong Polytechnic University Animal Subjects Ethics Review (22-23/479-OTHERS-R-OTHERS). This animal study was performed at the Hong Kong Polytechnic University Shenzhen Research Institute animal facility.

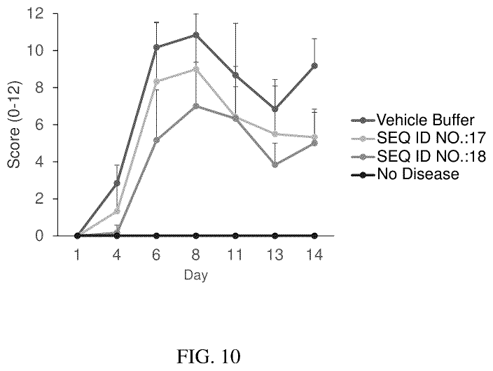

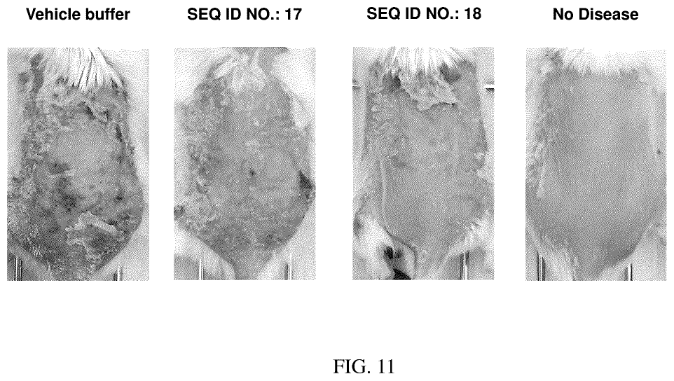

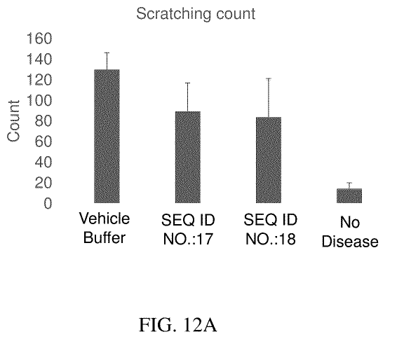

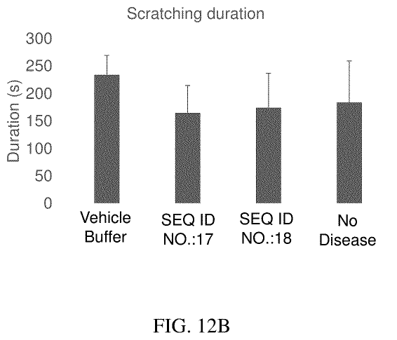

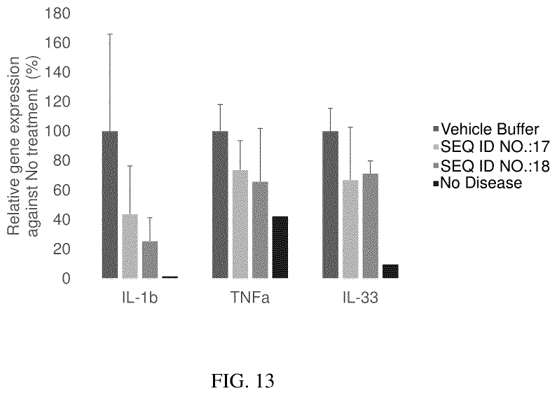

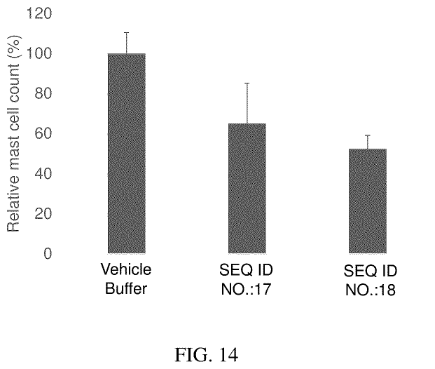

On Day 0, the dorsal skin had its fur removed. On Day 1, the shaved skin was measured for skin thickness and evaluated for Eczema Area Severity Index (EASI) scoring. The mice were then given vehicle buffer or 600 pmol of the aptamer in 100 μL. 100 of DNFB (0.2% v/v in acetone: olive oil (4:1)) was applied onto the back skin. For Days 4, 6, 8, 11, 13, the administration of the test-article and DNFB solution was repeated. On Days 6, 30 minutes after DNFB application, the mice were filmed for 15 minutes for the purpose of evaluating scratch response duration and frequency. On Day 14, the dorsal skin thickness was measured, and the skin was evaluated for EASI scoring. The mice were then sacrificed, and tissue samples were collected. Skin was wither fixed in 4% PFA or stored in RNA later for later TRIzol RNA extraction and qPCR. When mice were given SEQ ID NO.: 17 or SEQ ID NO.: 18 as a subcutaneous treatment option in conjunction with DNFB solution, the aptamer sequences inhibited the development of the atopic dermatitis phenotype (FIG. 10). FIG. 11 showed representative pictures of mouse dorsal skins from various treatment groups. During the 15 min observation on Day 6, mice treated with SEQ ID NO.: 17 or 18 were having less scratch count and shorter starching duration compared to that of vehicle control (FIG. 12A and FIG. 12B). qPCR analysis of the RNA samples collected at the end of the experiment showed that treatment of SEQ ID NO.: 17 or 18 could reduce the expression levels of proinflammatory cytokines IL-1b, TNFα and IL-33 (FIG. 13). Mast cells play a pivotal role in eczema progression, therefore the dorsal skin sections collected at the end of the experiment were stained with toluidine blue and examined for mast cells. FIG. 14 showed the percentage of mast cell count relative to vehicle buffer.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

What is claimed is:

1. An aptamer, wherein the aptamer comprises a nucleotide sequence with a sequence identity of at least about 80%, 85%, 90%, 95% or 100% sequence identity to any one of SEQ ID NOS.: 1-18, or a variant or a fragment thereof.

2. The aptamer of claim 1, wherein the nucleotide sequence comprises or consist of SEQ ID NO.: 3, SEQ ID NO.: 6, SEQ ID NO.: 16, SEQ ID NO.: 17 or SEQ ID No.: 18.

3. The aptamer of claim 1, wherein the nucleotide sequence comprises one or more of the following nucleoside substitutions: uracil, inverted dT, purine, xanthine, diaminopurine, 8-oxo-N6-methyladenine, 7-deazaxanthine, 7-deazaguanine, N4,N4-ethanocytosin, N6,N6-ethano-2,6-diaminopurine, 5-methylcytosine, 5-(C3-C6)-alkynylcytosine, 5-fluorouracil, 5-bromouracil, pseudoisocytosine, 2-hydroxy-5-methyl-4-triazolopyridin, isocytosine, isoguanine, inosine, non-naturally occurring nucleobases, variants, mutants, or analogs thereof.

4. The aptamer of claim 1, wherein the nucleotide sequence comprises one or more of the following sugar phosphate backbone modifications: 2′-O-methylation, locked nucleic (LNA), peptide nucleic acids (PNA), phosphorothioate, boranophosphate, methyl phosphate, 2′fluoro, 2′-O-methoxyethyl, or 4′-thio.

5. The aptamer of claim 1, wherein the nucleotide sequence is conjugated to one or more of the following chemical structures: cholesterol, cholesteryl, inverted dT, phosphatidyl choline, phosphatidyl ethanolamine, spermine, sialic acid, histidyl poly(lysine), palmityl-D-glucuronide, polyethylene glycol, polyrotaxanes, chlorogenic acid chitosan, listeriolysin O, or tyloxapol.

6. The aptamer of claim 1, wherein the nucleotide sequence is conjugated to the one or more chemical structures through one or more of the following spacers or linkers: C12 spacer, C9 spacer, C6 spacer, C3 spacer, spacer 18 (hexaethylene glycol), spacer 9, dSpacer, rSpacer, amino linkers, carboxy linkers, or thiol linkers.

7. The aptamer of claim 1, wherein the nucleotide sequence comprises the following modifications: a substitution of cytosines with 2′-O-methylation, and an addition of an inverted dT at the 3′ terminus.

8. The aptamer of claim 1, wherein the nucleotide sequence comprises the following modifications: a substitution conjugation of a cholesteryl at the 5′ terminus through a spacer C9 and an inverted dT at the 3′ terminus.

9. A pharmaceutical composition, comprising:
the aptamer of claim 1 or claim 2; and
optionally, a pharmaceutically acceptable carrier.

10. A method for preventing or treating BCMA-associated diseases or conditions, comprising:
administering an effective amount of an aptamer or a composition thereof, wherein the aptamer comprises a nucleotide sequence with a sequence identity of at least about 80%, 85%, 90%, 95% or 100% sequence identity to any one of SEQ ID NOS.: 1-18, or a variant or a fragment thereof, to a subject in need thereof.

11. The method of claim 10, wherein the nucleotide sequence comprises or consist of SEQ ID NO.: 3, SEQ ID NO.: 6, SEQ ID NO.: 16, SEQ ID NO.: 17 or SEQ ID No.: 18.

12. The method of claim 10, wherein the composition comprises a pharmaceutically acceptable carrier.

13. The method of claim 10, wherein BCMA-associated diseases or conditions are chronic inflammatory skin diseases, disorders, or conditions.

14. The method of claim 10, wherein the chronic inflammatory skin diseases, disorders, or conditions is selected from a group consisting of eczema and psoriasis.

15. The method of claim 10, wherein the aptamer or the composition thereof is administered by subcutaneous injection.

16. The method of claim 10, further comprising the step of: topically administering an effective amount of imiquimod (IMQ) after the step of administering an effective amount of an aptamer or a composition thereof.


SEQUENCE LISTING (18 sequences)

SEQ ID NO: 1 - DNA, 71 bp, synthetic construct
  source 1..71: mol_type = other DNA
cgtacggtcgacgctagcaagtatagacgtgatgcagggaatgcagacccaggggatccgagctccacgtg

SEQ ID NO: 2 - DNA, 71 bp, synthetic construct
  source 1..71: mol_type = other DNA
cgtacggtcgacgctagcccatgggaatgcagaccgcaggctggctgtaggagggatccgagctccacgtg

SEQ ID NO: 3 - DNA, 71 bp, synthetic construct
  source 1..71: mol_type = other DNA
cgtacggtcgacgctagctgttgctgtgcaggaagaatgcagattcccaacatggatccgagctccacgtg

SEQ ID NO: 4 - DNA, 35 bp, synthetic construct
  source 1..35: mol_type = other DNA
cgtacggtcgacgctagctgttgctgtgcaggaag

SEQ ID NO: 5 - DNA, 41 bp, synthetic construct
  source 1..41: mol_type = other DNA
cgtacggtcgacgctagctgttgctgtgcaggaagaatgca

SEQ ID NO: 6 - DNA, 46 bp, synthetic construct
  source 1..46: mol_type = other DNA
cgtacggtcgacgctagctgttgctgtgcaggaagaatgcagattc

SEQ ID NO: 7 - DNA, 51 bp, synthetic construct
  source 1..51: mol_type = other DNA
cgtacggtcgacgctagctgttgctgtgcaggaagaatgcagattcccaac

SEQ ID NO: 8 - DNA, 56 bp, synthetic construct
  source 1..56: mol_type = other DNA
cgtacggtcgacgctagctgttgctgtgcaggaagaatgcagattcccaacatgga

SEQ ID NO: 9 - DNA, 61 bp, synthetic construct
  source 1..61: mol_type = other DNA
cgtacggtcgacgctagctgttgctgtgcaggaagaatgcagattcccaacatggatccga

SEQ ID NO: 10 - DNA, 66 bp, synthetic construct
  source 1..66: mol_type = other DNA
cgtacggtcgacgctagctgttgctgtgcaggaagaatgcagattcccaacatggatccgagctcc

SEQ ID NO: 11 - DNA, 35 bp, synthetic construct
  source 1..35: mol_type = other DNA
tgttgctgtgcaggaagaatgcagattcccaacat

SEQ ID NO: 12 - DNA, 66 bp, synthetic construct
  source 1..66: mol_type = other DNA
ggtcgacgctagctgttgctgtgcaggaagaatgcagattcccaacatggatccgagctccacgtg

SEQ ID NO: 13 - DNA, 61 bp, synthetic construct
  source 1..61: mol_type = other DNA
acgctagctgttgctgtgcaggaagaatgcagattcccaacatggatccgagctccacgtg

SEQ ID NO: 14 - DNA, 56 bp, synthetic construct
  source 1..56: mol_type = other DNA
agctgttgctgtgcaggaagaatgcagattcccaacatggatccgagctccacgtg

SEQ ID NO: 15 - DNA, 51 bp, synthetic construct
  source 1..51: mol_type = other DNA
ttgctgtgcaggaagaatgcagattcccaacatggatccgagctccacgtg

SEQ ID NO: 16 - DNA, 47 bp, synthetic construct
  source 1..47: mol_type = other DNA
  modified_base 1: mod_base = OTHER; note = 2 O methylation
  modified_base 5: mod_base = OTHER; note = 2 O methylation
  modified_base 9: mod_base = OTHER; note = 2 O methylation
  modified_base 12: mod_base = OTHER; note = 2 O methylation
  modified_base 14: mod_base = OTHER; note = 2 O methylation
  modified_base 18: mod_base = OTHER; note = 2 O methylation
  modified_base 24: mod_base = OTHER; note = 2 O methylation
  modified_base 29: mod_base = OTHER; note = 2 O methylation
  modified_base 40: mod_base = OTHER; note = 2 O methylation
  modified_base 46: mod_base = OTHER; note = 2 O methylation
  modified_base 47: mod_base = OTHER; note = 3 Inverted dT
cgtacggtcgacgctagctgttgctgtgcaggaagaatgcagattct

SEQ ID NO: 17 - skipped

SEQ ID NO: 18 - DNA, 47 bp, synthetic construct; identical to SEQ ID NO: 16
  source 1..47: mol_type = other DNA
  modified_base 47: mod_base = OTHER; note = 3 Inverted dT
  misc_feature 1: note = the first residue is attached through linker C9 to a cholesteryl